(19) United States
(12) Patent Application Publication
(10) Pub. No.: US 2008/0045995 A1
(43) Pub. Date: Feb. 21, 2008

(54) ANEURYSM BUTTRESS ARRANGEMENT

(76) Inventors: Lee R. GUTERMAN, Amherst, NY (US); Paul A. LaDUCA, Buffalo, NY (US); Robert C. LaDUCA, Santa Cruz, CA (US)

Correspondence Address: LEVINE BAGADE HAN LLP, 2483 EAST BAYSHORE ROAD, SUITE 100, PALO ALTO, CA, 94303, US

(21) Appl. No.: 11/844,323

(22) Filed: Aug. 23, 2007

Related U.S. Application Data
(63) Continuation of application No. PCT/US2005/005718, filed on Feb. 23, 2005.

(51) Int. Cl. A61M 29/02

(52) U.S. Cl. 606/194; 606/200

(57) ABSTRACT

An aneurysm buttressing arrangement for covering an aneurysm opening in an intracranial aneurysm, for temporary placement thereadjacent, to prevent escape of embolitic agents from that aneurysm. The arrangement comprises an elongated delivery wire having a proximal end and a tracking distal end wire, a scaffold of expandable wires arranged proximal to and in spaced adjacent relationship to the distal end of the delivery wire, wherein the scaffold of wires has a tapered proximal end and a tapered distalmost end, the scaffold being expandable upon deployment from a delivery catheter, and collapsible for withdrawal into a delivery catheter, the tracking distal end wire extending distally from the scaffold about one-half to about ten centimeters.

### BACKGROUND INFORMATION

The present invention relates to the treatment of vascular aneurysms, and, in particular, to methods and devices for filling aneurysms with an embolic or other material, while maintaining patency of the adjacent vessel.

Various implantable medical devices have been developed for treating ailments in the vascular system. Vaso-occlusive devices have been used extensively in closing regions of the vascular system. These devices are especially useful in treating aneurysms. Vascular aneurysms are formed as a result of a weakening in the wall of an artery and subsequent ballooning of the artery wall. Aneurysms are often a site of internal bleeding and, catastrophically, result in hemorrhagic strokes. A variety of different embolic agents are known to be suitable for treatment of such aneurysms. These treatments are commonly known as “artificial vaso-occlusion.”

Recent advancements in the artificial occlusion of vessels and aneurysms have occurred mostly due to the improvements in delivery and implantation of metal coils as vaso-occlusive devices. Implantable metal coils that are useful in artificial occlusion devices in vasculature lumens or aneurysms are herein referred to as “vaso-occlusive coils”.

Vaso-occlusive coils are generally constructed of wire, usually made of a metal or metal alloy, that is first wound into a shape such as a sphere or helix. Many such devices are introduced to the selected target site through a catheter in a stretched linear form. The vaso-occlusive device may assume an irregular shape upon discharge of the device from the distal end of the catheter. A variety of vaso-occlusive coils and braids are known. For instance, U.S. Pat. No. 4,994,069, to Ritchart et al., shows a flexible, preferably coiled, wire for use in small vessel vaso-occlusion. Theses coils are described as being between 0.010 and 0.030 inches in diameter. The wire used to make up the coils may be, for instance, 0.002 to 0.006 inches in diameter. Tungsten, platinum, and gold threads or wires are said to be preferred. These devices may be used to fill aneurysms.

It is common for these vaso-occlusive devices to be delivered through microcatheters such as the type shown in U.S. Pat. No. 4,739,768, to Engelson. These microcatheters track a guidewire to a point just proximal or within the desired occlusion site. The vaso-occlusive coils are then advanced through the microcatheter, once the guidewire is removed, and out the distal end hole so to at least partially fill the selected site and create occlusion within the aneurysm. Experiments have indicated that, at most, 40% of the aneurysm is filled with coils. The remainder is filled with naturally occurring thrombus.

In addition to the various types of space-filling mechanisms and geometries of vaso-occlusive coils, other particularized features of coil designs, such as mechanisms for delivering vaso-occlusive coils through delivery catheters and implanting them in desired occlusion sites, have also been described. Examples of such vaso-occlusive devices based upon their delivery mechanisms include pushable coils (Ritchart et al., discussed above), mechanically detachable vaso-occlusive devices (U.S. Pat. No. 5,261,916 to Engelson or U.S. Pat. No. 5,250,071 to Palermo), or electrolytically detachable vaso-occlusive devices (U.S. Pat. Nos. 5,122,136 and 5,354,295 to Guglielmi et al.). Other prior art such as U.S. Pat. No. 5,916,235 to Guglielmi discloses methods and apparatus which have similar characteristics and limitations for not facilitating buttressing, delivery or tracking or the like.

However, after, or perhaps during, delivery of such a coil into the aneurysm, there is a risk that a portion of the coil might migrate out of the aneurysm entrance zone and into the feeding vessel. This is especially true in aneurysms where the diameter of the aneurysm neck approaches the diameter of the aneurysm body in a 1:1 ratio. The presence of such a coil in that feeding vessel may cause the undesirable response of causing an occlusion there. Also, there is a quantifiable risk that the blood flow in the vessel and the aneurysm may induce movement of the coil farther out of the aneurysm, resulting in a more thoroughly developed embolus in the parent vessel. Being that coils are constructed from very low gauge wire, the coil mass can compact resulting in aneurysm recanalization.

Furthermore, one type of aneurysm, commonly known as a “wide-neck aneurysm” is known to present particular difficulty in the placement and retention of vaso-occlusive coils. Wide-neck aneurysms are herein referred to as aneurysms of vessel walls having a neck or an “entrance zone” from the adjacent vessel, which entrance zone has a diameter of either (1) at least 80% of the largest diameter of the aneurysm; or (2) is clinically observed to be too wide effectively to retain vaso-occlusive coils that are deployed using the techniques discussed herein.

Vaso-occlusive coils lacking substantial secondary shape strength may also be difficult to maintain in position within an aneurysm no matter how skillfully they are placed. This may also be true of coils that have a secondary shape. For example, a 3D coil that takes a spherical shape may be herniated out of the aneurysm into the parent vessel if the neck is too wide. Using the buttressing device of the present invention permits the coils to be held in the aneurysm until a critical mass of coils is achieved within the aneurysm so that the coil mass will not move when the buttressing device is withdrawn.

A few devices have been disclosed for maintaining the presence of vaso-occlusive coils within an aneurysm. One such device is a retainer for retaining coils within the aneurysm cavity. The retainer device is released into the vessel exterior to the aneurysm. The device is held in place via the presence of radial pressure on the vessel wall. After the device is released and set in an appropriate place. A microcatheter is inserted into the lumen so that the distal end of the catheter is inserted into the aneurysm cavity. One or more vaso-occlusive devices is then introduced into the aneurysm cavity. The retainer device maintains the presence of the vaso-occlusive devices within the aneurysm whether it is a large-mouth aneurysm or not.

Another approach to filling intracranial aneurysms includes the use of injectable fluids or suspensions, such as microfibrillar collagen, various polymeric beads, and polyvinyl alcohol foam. These polymeric agents may additionally be crosslinked, sometimes in vivo to extend the persistence of the agent at the vascular site. These agents may be introduced into the vasculature through any of a variety of known catheters. After introduction, the deployed materials form a solid space-filling mass. Other materials, including polymeric resins, typically cyanoacrylates, hydrogels and other gels, fibrin glues, and calcium binding seaweed extracts are also employed as injectable vaso-occlusive materials. These materials may be mixed with a radio-opaque contrast material or made radio-opaque by the addition of a tantalum powder.

The delivery of liquid embolic agents into aneurysms in general has numerous obstacles. The viscosity of the material makes delivery difficult, and leads to run on even after the pressure head has been removed from the delivery catheter. Inadequate opacification of the material makes it difficult to see. As a result it can leak into the parent vessel. This can result in vessel occlusion and distal embolization into the organs vascular bed. To date, these materials have been delivered using an inflated balloon adjacent to the abnormality to be treated. Inflation of the balloon during delivery leads to temporary vessel occlusion and can result in downstream organ ischemia and even infarction.

Thus, notwithstanding the various efforts in the prior art, there remains a need for an embolic deployment system which enables the filling and sealing of an aneurysm while minimizing the risk of leakage and subsequent migration of any material delivered into the aneurysm, and enabling perfusion during the deployment process.

### SUMMARY

The present invention relates to a method of filling and buttressing an intracranial aneurysm. The method comprises the steps of transluminally positioning a buttress scaffold across an opening of an aneurysm in an intracranial vessel so as to block off and isolate that aneurysm cavity in a side wall of that vessel. Media such as embolitic agents, coils, and or polymers may then be introduced into that cavity within the sidewall of the vessel. The cavity is often of a bulbous shape having a neck portion of no greater than about one-half the diameter of the bulbous.

The buttress scaffold is arranged on the distal end of an elongated “delivery” wire or “pushwire”, much like a guide wire. The scaffolding itself may be comprised of a braid of wire, comprised of a memory metal or polymeric fibers and or plastic, or a co-weave combination thereof. The proximal end of the scaffold has a taper and the distal end of the scaffolds also has a taper. A tracking tip is arranged on the distal end of the scaffold and has a length of about one-half centimeter to about ten centimeters extending therefrom. The buttress scaffold may in an alternative embodiment, be comprised of a generally cylindrically shaped array of helically wound wires which expand into a diameter of between two and six millimeters from an unexpanded diameter of generally about 0.020 inches, which buttress scaffold and delivery wire/pushwire is arranged through a microcatheter having an internal diameter generally about 0.018 to about 0.025 inches.

The buttress scaffold arrangement is introduced adjacent the aneurysm through a microcatheter pushed into the subject parent vessel, having its distalmost end placed adjacent the neck of the aneurysm. The distal tracking tip on the distal end of the scaffold may assist in directing that scaffold further downstream in the parent vessel distal of the microcatheter which delivered it. As the buttress scaffold is preferably delivered adjacent the neck of the aneurysm, it is permitted to expand to the diameter of the parent vessel.

A further microcatheter may be introduced either or both alongside or through an internal lumen of the delivery wire/pushwire delivering the buttress scaffold so as to also permit the introduction of an embolitic agent into the aneurysm through, around or adjacent the mesh of the buttress scaffold. That mesh of the buttress scaffolding, whether it is a braided or a helical arrangement, preferably has opened spaces or cells which permit that microcatheter and delivery wire to introduce that embolitic agent into the aneurysm. Such an agent may be comprised of metallic or plastic coils, or alternatively a combination of plastic and metal braid or composite plastic and metal braid and/or liquid or polymerized polymeric agents, or biologic components of blood and plasma like thrombin, fibrin or any biologic materials like DNA, RNA plasmids or the like, to embolize within that aneurysm.

A further embodiment of the buttress scaffold of the present invention is comprised of a plurality of layers of helically wound wires defining that mesh. The distal end of that scaffold being sloped into and attached to the extended distal tracking tip to help facilitate steering of that scaffolding within the parent vessel.

A further embodiment of the present invention contemplates the pushwire at the proximal end of the buttress scaffold to be hollow, with a thin control wire extending therethrough. The control wire is elongated and extends out the proximalmost end of the scaffold pushwire. The control wire has a distalmost end fixed to the distalmost end of the scaffold. The control wire may be moved longitudinally relative to the delivery wire/pushwire of the scaffold. Movement of the control wire relative to the delivery wire/pushwire permits dimensional control of the scaffold and facilitates advance of the distal tracking tip of the scaffold within the parent vessel. The internal control wire within the delivery wire/pushwire of the scaffold may be rotated about its longitudinal axis so as to effect rotation of the scaffold or a winding thereof relative to the pushwire so as to effect radial and/or longitudinal dimensional changes of that scaffold depending upon the “handedness” of the helical coil or braid making up that scaffold.

A further embodiment of that scaffold, comprises at least a portion of the cylindrical section thereof which may be wrapped within a thin polymeric film to facilitate movement of that scaffold within the parent vessel or to enhance the buttressing effect of that scaffold adjacent the neck opening of the aneurysm. The cells defining the mesh and any polymeric film would be pierceable by the adjacent microcatheter delivery wire advancing into the aneurysm itself. The film may also be foraminous, to permit a microcatheter or medicaments to be delivered therethrough. The film also facilitates delivery of a microcatheter around the outside thereof and into the aneurysm.

In a still further embodiment of the present invention, the pushwire on the proximal end of the buttress scaffold is hollow, and contains on its distalmost end within that scaffold, a thin elongated balloon in fluid communication with the lumen in that hollow delivery wire/pushwire. Such a combination permits the buttress scaffold to be expanded to the diameter of the parent vessel by inflation of that balloon within that scaffold through a pressurized fluid introduced through the lumen within the hollow pushwire. Deflation of that balloon in a periodic manner would permit blood flow through the parent vessel while also permitting introduction of an embolitic agent into that aneurysm. The balloon may be pressurized and depressurized by a liquid medicament for subsequent intended release of that medicament treatment of the situs at the neck of the aneurysm, by a puncture of that balloon and control of the pressure therewithin by a pressure control means at the proximal end of the hollow pushwire.

The scaffold, by virtue of its tapered proximalmost end is permitted to be withdrawn into the distalmost opening of the micro delivery catheter from which was introduced.

The invention thus comprises an aneurysm buttressing arrangement for covering an aneurysm opening in an intracranial aneurysm, for temporary placement thereadjacent, to prevent escape of embolitic agents from that aneurysm. The arrangement comprises an elongated delivery wire having a proximal end and a tracking distal end wire; a scaffold of expandable wires arranged proximal to and in spaced adjacent relationship to the distal end of the delivery wire, wherein the scaffold of wires has a tapered proximal end and a tapered distalmost end wire, the scaffold being expandable upon deployment from a delivery catheter, and collapsible for withdrawal back into a delivery catheter; the tracking distal end wire extending distally from the scaffold about one-half to about ten centimeters. The elongated delivery wire may be hollow. The scaffold is preferably comprised of a collection of circumferentially spaced helically directed wires. The circumferentially spaced helically directed wires may be comprised of at least two layers thereof. The scaffold may also comprised of a braided array or a combination of braided and helical metal and or plastic wires. The expandable wires define open cells therebetween, for blood flow therethrough and sized to prevent herniation of embolitic agents from the aneurysm. The scaffold may be detachable from the elongated wire. The elongated hollow delivery wire may have a control wire extending centrally therethrough, the control wire extending up through and fixedly attached to the distal end of the scaffold. The control wire has a distalmost end which in one preferred embodiment may comprise the tracking distal end wire. The scaffold may have a pierceable or foraminous film disposed therearound. An inflatable and deflatable elongated balloon may be arranged within the scaffold. The balloon may be pressurized and depressurized by a fluid transmitted through the hollow delivery wire to the balloon. The fluid may be a liquid medicament which may be pierced by a microcatheter to facilitate delivery of that liquid.

The invention may also comprise a method of buttressing an intracranial aneurysm in a vessel wall, comprising the steps of: transluminally positioning a scaffold out of a delivery catheter, the scaffold having a proximal end and a distal end arranged onto a near distal of a delivery wire across the opening of an aneurysm; expanding the scaffold from a contracted diameter to engage the vessel wall by a spacing open of helically wound wire coils comprising the scaffold; introducing an embolitic agent into the aneurysm through a cell between adjacent wires comprising the coils; permitting blood to flow through the cells of the scaffold subsequent to the introduction of the embolitic agent into the aneurysm; and

withdrawing the scaffold from its position adjacent the aneurysm. The method may also comprise one or more of the following steps of: placing a thin film about the scaffold prior to positioning of the scaffold adjacent the aneurysm; inserting a balloon within the scaffold prior to positioning of the scaffold adjacent the aneurysm; arranging the delivery wire to have a central lumen therethrough; placing a control wire through the lumen in the delivery wire; extending the control wire through the scaffold distally; and attaching the control wire to a distalmost end of the scaffold; extending the control wire distally of the scaffold so as to function as a distal tracking wire; moving the control wire so as to vary the size and shape of the scaffold; tapering the distal and proximal ends of the scaffold to facilitate sliding of the scaffold out of and back into the delivery catheter.

The wire incorporated into the distal portion of the device of the present invention, including the braided portion thereof may in a further embodiment, be constructed of a composite material to increase the radio-opacity of the device. For example, Nitinol filled with a radio-opaque metal such as tantalum, platinum or stainless steel filled with platinum, tantalum or tungsten.

### BRIEF DESCRIPTION OF THE DRAWINGS

The objects and advantages of the present invention will become more apparent, when viewed in conjunction with the following drawings in which:

FIG. 1 is a side elevational view of a micro delivery catheter and buttress scaffold arrangement being introduced into an intracranial vessel, adjacent an aneurysm therein;

FIG. 2 is a side elevational view similar to FIG. 1, showing the buttress scaffold arrangement positioned adjacent the neck opening of an aneurysm in a sidewall of that intracranial vessel;

FIG. 3 is a side elevational view of the buttress scaffold arrangement adjacent the aneurysm, with a further microcatheter alongside, utilized for introducing an embolitic agent into that aneurysm;

FIG. 4 is a side elevational view of a first embodiment of the buttress scaffold arrangement;

FIG. 5 is a further embodiment of the buttress scaffold arrangement of the present invention;

FIG. 6 is a yet further embodiment of the buttress scaffold arrangement of the present invention; and

FIG. 7 is yet still a further embodiment of the buttress scaffold arrangement of the present invention.

### DETAILED DESCRIPTION OF THE PREFERRED EMBODIMENTS

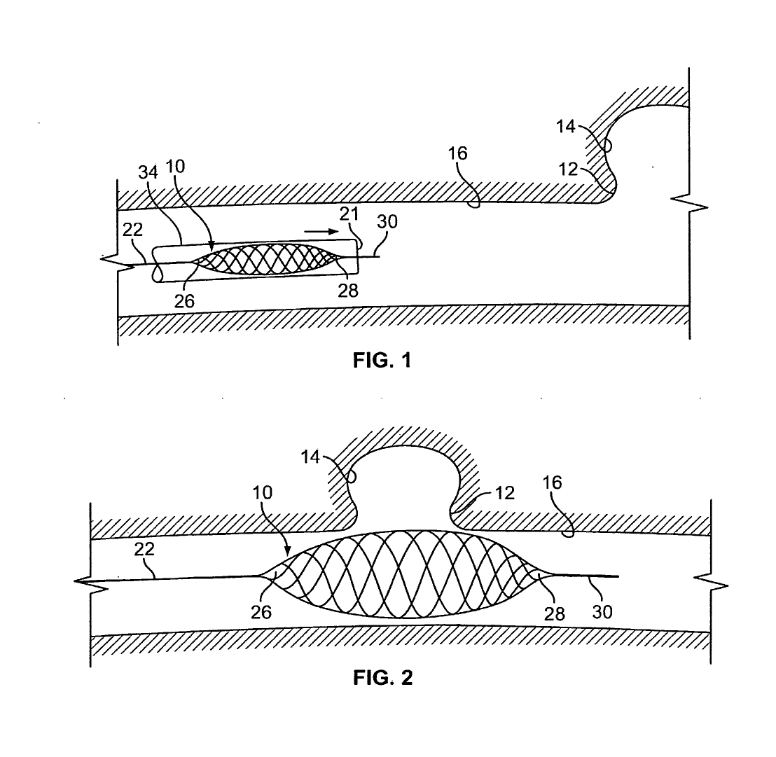

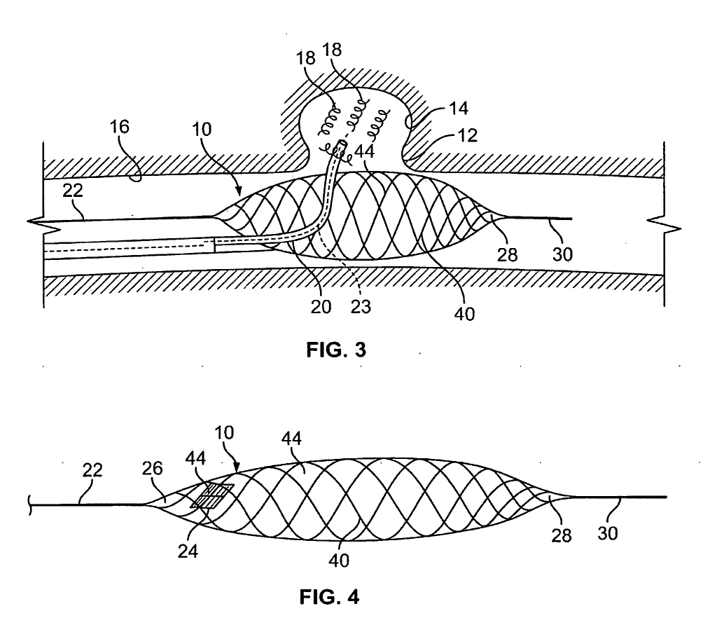

Referring now to the drawings in detail, and particularly to FIG. 1, there is shown the present invention which comprises a method of filling and buttressing an intracranial aneurysm. The method comprises the steps of transluminally positioning a buttress scaffold 10 from an initial unexpanded delivery diameter of for example about 0.018 to about 0.030 inches into an expanded diameter of for example about 2-6 mm across an opening 12 of an aneurysm 14 in an intracranial vessel 16 so as to block off and isolate that aneurysm cavity 14 in a side wall of that vessel 16, as shown in FIGS. 1, 2 and 3. Media such as embolitic agents 18, for example, coils, and or polymers may then be introduced by a further microcatheter 20, into that cavity 14 within the sidewall of the vessel 16, as represented in FIG. 3. The cavity 14 for our description of treatment purposes is of a bulbous shape having a neck portion 12.

The buttress scaffold 10 is arranged on the distal end of an elongated wire 22 much like a guide wire, and may have a length of 185 cm or more. The scaffolding 10 itself may be comprised in one preferred embodiment thereof, of a braided cylinder 24 comprised of wire, as shown partially in FIG. 4, which wire may be comprised of a memory metal or plastic. The proximal end 26 of the scaffold 10, as represented in FIGS. 1-7, all have a taper. The distal end 28 of each scaffold 10 represented, also has a tapered configuration. An extended tracking tip wire 30 is arranged on the distal end 28 of the scaffold 10 and has a length of about one-half centimeter to about ten centimeters extending therefrom.

The buttress scaffold 10, may in an alternative embodiment, as represented in FIG. 4, be comprised of a generally cylindrically shaped array of helically wound wires 32 which expand into a diameter of between three and five millimeters from an unexpanded diameter of about 0.018-0.030 inches in the delivery microcatheter catheter 34.

The buttress scaffold arrangement is introduced adjacent the aneurysm 14 through the microcatheter 34, as represented in FIG. 1, and is pushed into the subject vessel, having its distalmost end 28 adjacent the neck 12 of the aneurysm 14. The distal tracking tip wire 30 on the distal end 28 of the scaffold 10 directs that scaffold 10 downstream in the vessel 16, distal of the microcatheter 34 which delivered it. As the buttress scaffold 10 is adjacent the neck 12 of the aneurysm it is thus permitted to expand to the diameter of the parent vessel 16, as represented in FIG. 2, once it is free of the confines of the delivery catheter 34.

A further microcatheter is preferably introduced alongside the pushwire 22 which wire 22 is proximally attached to the buttress scaffold 10, as represented in FIG. 3, so as to permit the introduction of an ambolitic agent 18 into the aneurysm 14 through or around the outside of the sidewall mesh 40 of the buttress scaffold 10. That mesh 40 of the buttress scaffold 10, whether it is a braided wire 24 or an arrangement helically wound wires 42, or a combination as recited hereinabove, as also represented in FIG. 4, have opened spaces or cells 44, of a dimension for example, of from about 500 microns to about 1 cm., which cells 44 permit a microcatheter 20 and its delivery wire 23 to pass unimpeded therethrough and subsequently introduce an embolitic agent 18 into the aneurysm 14. Such an agent 18 may be comprised of, for example, metallic or plastic coils, and or polymeric agents to embolize the media within that aneurysm 14. Those cells 44 are sized to prevent herniation by embolitic agents 18 such as coils, from escaping the aneurysm 14, while permitting blood to flow unimpeded through the parent vessel 16.

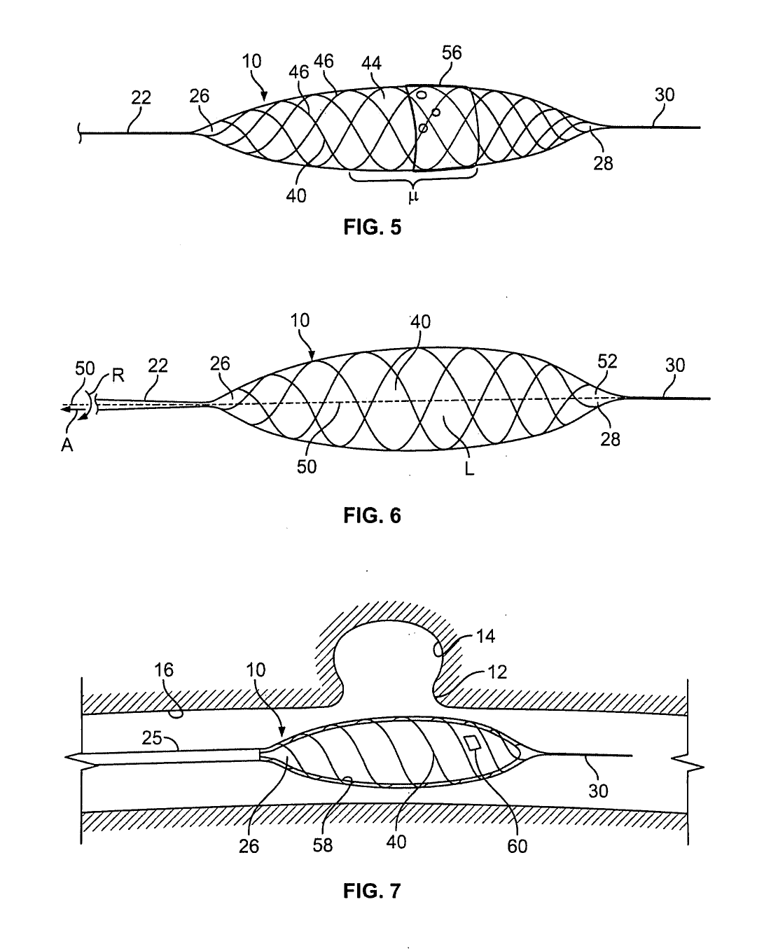

A further embodiment of the buttress scaffold 10 of the present invention is comprised of a plurality of radially adjacent layers of helically wound wires 46 defining that mesh 40, as represented in FIG. 5. The distal end 28 of that scaffold 10 is preferably sloped into the distal tracking tip 30 to permit steering of that scaffold 10 within the parent vessel 16.

A further embodiment of the present invention contemplates a delivery wire/pushwire 25 at the proximal end of the buttress scaffold 10 to be hollow, with a thin control wire 50 extending therethrough, as represented in FIG. 6. The control wire 50 is elongated, may be hollow itself for further delivery of medicaments or another microcatheter, and extends out the proximalmost end of the hollow pushwire 25 which itself is attached to the proximal end 26 of the scaffold 10. The control wire 50 has a distalmost end 52 fixed to the distalmost end 28 of the scaffold 10. The control wire 50 may be moved longitudinally relative to the delivery wire/pushwire 25 connected to the scaffold 10, as represented by arrow “A” in FIG. 6. Movement of the control wire 50 relative to the hollow pushwire 22 permits length and diametric dimensional control of the scaffold 10 and also facilitates advance of the distal tracking tip of the scaffold 10 within the parent vessel 16. The internal control wire 50 within the hollow delivery wire/pushwire 25 attached to the scaffold 10 my also be rotated about its longitudinal axis, as represented by arrow “R” so as to effect a twisting rotation of the scaffold 10 around its longitudinal axis “L”, or a winding thereof relative to the hollow pushwire 25 so as to effect longitudinal or diametric dimensional changes of that scaffold 10 depending upon the “handedness” of the helical coil 40 or braid 24 making up that scaffold 10.

A further embodiment of that scaffold 10, as represented partially in FIG. 5, comprises at least a portion of the middle or cylindrical section “M” thereof which may be wrapped within a thin elastomeric or polymeric film 56 which film 56 may be foraminous, to facilitate “covered” movement of that scaffold 10 within the parent vessel 16 or to enhance the buttressing effect of that scaffold 10 adjacent the neck opening 12 of the aneurysm 14. It is to be noted that stents placed in body vessels do not have such ability to be moved subsequent to placement within such a body vessel. The cells 44 defining the mesh 40 and any elastomeric/polymeric film 56 surround the cells 44 would be pierceable by an adjacent microcatheter delivery wire 23 advancing into the aneurysm 14 itself.

In a still further embodiment of the present invention as represented in FIG. 7, the delivery wire/pushwire 25 on the tapered proximal end 26 of the buttress scaffold 10 is hollow, and contains on its distalmost end within that scaffold 10, a thin elongated balloon 58 in fluid communication with the central lumen in that hollow pushwire 25. Such a combination permits the buttress scaffold 10 to be expanded to the diameter of the parent vessel 16 by inflation of that balloon 58 within that scaffold 10 through a pressurized fluid introduced through the lumen within the hollow pushwire 25. Deflation of that balloon 58, by proper controlled inflation/deflation means at the proximal end of the pushwire 25, not shown for clarity, in a periodic manner would permit blood flow through the parent vessel 16, while also permitting introduction of an embolitic agent 18 into that aneurysm 14. The balloon 58 may in a still further embodiment, be pressurized and depressurized by a pressure controlled liquid medicament for subsequent treatment of the situs at the neck 12 of the aneurysm 14, by a fluid release means 60 such as piercing by a further microcatheter, on/in/through the balloon 58.

The scaffold 10, by virtue of its tapered proximalmost end 26 is permitted to be withdrawn into the distalmost opening 21 of the micro delivery catheter 34 from which was introduced, as represented in FIG. 1.

1. (canceled)

2. (canceled)

3. The aneurysm buttressing arrangement as recited in claim 25, wherein said scaffold is comprised of a collection of circumferentially spaced helically directed wires.

4. The aneurysm buttressing arrangement as recited in claim 3, wherein said circumferentially spaced helically directed wires have at least two layers thereof.

5. The aneurysm buttressing arrangement as recited in claim 25, wherein said scaffold is comprised of a braided array of wires.

6. The aneurysm buttressing arrangement as recited in claim 25, wherein said expandable wires define open cells therebetween, for blood flow therethrough and sized to prevent herniation of embolitic agents from said aneurysm.

7. (canceled)

8. The aneurysm buttressing arrangement as recited in claim 25, wherein said elongated hollow delivery wire has a control wire extending therethrough, said control wire extending up through and fixedly attached to said distal end of said scaffold.

9. The aneurysm buttressing arrangement as recited in claim 8, wherein said control wire has a distal most end which comprises said tracking distal end wire.

10. The aneurysm buttressing arrangement as recited in claim 25, wherein said scaffold has a film disposed therearound.

11. (canceled)

12. The aneurysm buttressing arrangement as recited in claim 25, wherein said balloon is pressurized and depressurized by a fluid transmitted through said hollow delivery wire to said balloon.

13. The aneurysm buttressing arrangement as recited in claim 12, wherein said fluid is a liquid medicament.

14. The aneurysm buttressing arrangement as recited in claim 10, wherein said film is foraminous.

15. (canceled)

16. The method as recited in claim 27, comprising the step of:
placing a thin film about said scaffold prior to positioning of said scaffold adjacent said aneurysm to facilitate buttressing thereof.

17. (canceled)

18. (canceled)

19. (canceled)

20. The method as recited in claim 27, comprising the step of:
extending said control wire through said scaffold to a distalmost end thereof; and
attaching said control wire to said distalmost end of said scaffold.

21. The method as recited in claim 20, comprising the step of:
extending said control wire distally of said scaffold so as to function as a distal tracking wire.

22. The method as recited in claim 20, comprising the step of:
moving said control wire with respect to said delivery wire/pushwire so as to permit length and/or diametric dimension control of said scaffold.

23. The method as recited in claim 27, comprising the step of:
tapering said distal and proximal ends of said scaffold to facilitate sliding of said scaffold out of and back into said delivery catheter.

24. An aneurysm buttressing arrangement for covering an aneurysm opening in an intracranial aneurysm, for temporary placement there adjacent, to prevent escape of embolitic agents from that aneurysm, said arrangement comprising:
an elongated delivery wire having a proximal end and a tracking distal end wire;
a scaffold including a collection of circumferentially spaced helically directed expandable wires arranged proximal to and in spaced adjacent relationship to said distal end of said delivery wire, wherein said scaffold has a tapered proximal end and a tapered distalmost end, wherein said scaffold is expandable upon deployment from a delivery catheter, collapsible for withdrawal into a delivery catheter, and detachable from said elongated wire;
said tracking distal end extending distally from said scaffold one to three centimeters.

25. An aneurysm buttressing arrangement for covering an aneurysm opening in an intracranial aneurysm, for temporary placement there adjacent, to prevent escape of embolitic agents from that aneurysm, said arrangement comprising:
a hollow, elongated delivery wire having a proximal end and. a tracking distal end wire;
a scaffold of expandable wires arranged proximal to and in spaced adjacent relationship to said distal end of said delivery wire, wherein said scaffold of wires has a tapered proximal end and a tapered distalmost end, said scaffold being expandable upon deployment from a delivery catheter, and collapsible for withdrawal into a delivery catheter;
said tracking distal end extending distally from said scaffold one to three centimeters; and an inflatable and deflatable elongated balloon disposed within said scaffold.

26. A method of buttressing an intracranial aneurysm in a vessel wall, comprising the steps of:
transluminally positioning a scaffold of wires out of a delivery catheter, said scaffold having a proximal end and a distal end arranged onto a near distal of a delivery wire across the opening of an aneurysm;
inserting a balloon within said scaffold expanding said scaffold from a first contracted diameter to a second diameter to engage said vessel wall by expanding a helically wound wire comprising said scaffold with said balloon;
introducing an embolitic agent into said aneurysm through a cell between adjacent wires comprising said scaffold;
permitting blood to flow through said cells of said scaffold subsequent to said introduction of said embolitic agent into said aneurysm; and
withdrawing said scaffold from its position adjacent said aneurysm.

27. A method of buttressing an intracranial aneurysm in a vessel wall, comprising the steps of:
transluminally positioning a scaffold of wires out of a delivery catheter, said scaffold having a proximal end and a distal end arranged onto a near distal of a delivery wire across the opening of an aneurysm, said delivery wire having a central lumen therethrough;
expanding said scaffold from a first contracted diameter to a second diameter to engage said vessel wall by expanding a helically wound wire comprising said scaffold;
placing a control wire through said lumen in said delivery wire;
introducing an embolitic agent into said aneurysm through a cell between adjacent wires comprising said scaffold;
permitting blood to flow through said cells of said scaffold subsequent to said introduction of said embolitic agent into said aneurysm; and
withdrawing said scaffold from its position adjacent said aneurysm.
